(19) United States
(12) Patent Application Publication
(10) Pub. No.: US 2024/0033685 A1
(43) Pub. Date: Feb. 1, 2024

(54) Direct Air Capture and Bioelectrochemical Conversion of CO2

(71) Applicant: The Regents of the University of California, Oakland, CA (US)

(72) Inventors: Douglas S. Clark, Berkeley, CA (US); Omar M. Yaghi, Berkeley, CA (US); Peidong Yang, Berkeley, CA (US); Anthony Abel, Berkeley, CA (US); Jeremy Adams, Berkeley, CA (US); Stefano Cestellos-Blanco, Berkeley, CA (US); Hao Lyu, Berkeley, CA (US); Ayan Zhumekenov, Berkeley, CA (US)

(73) Assignee: The Regents of the University of California, Oakland, CA (US)

(21) Appl. No.: 18/482,871

(22) Filed: Oct. 7, 2023

Related U.S. Application Data
(63) Continuation of application No. PCT/US22/27948, filed on May 5, 2022.
(60) Provisional application No. 63/187,862, filed on May 12, 2021.

(51) Int. Cl. B01D 53/84; B01D 53/14; C07C 29/151; C12M 1/00

(52) U.S. Cl. CPC B01D 53/84; B01D 53/1475; C07C 29/1518; C12M 21/12

(57) ABSTRACT

An integrated, modular system for direct air capture (DAC) and electro-microbial production (EMP) for bioelectrochemical conversion of CO₂ comprises (a) a solid absorbent configured to directly capture CO₂ from air; (b) a first bioreactor configured to receive enriched and purified CO₂ from the absorbent and convert the CO₂ to an upgradeable organic carbon intermediate by an autotrophic microorganism, wherein the autotrophic organism derives energy from oxidation of electrochemically-generated reducing equivalents; and (c) a second bioreactor configured to receive the organic carbon intermediate from the first bioreactor for use as a feedstock by a metabolically engineered microorganism to generate a value added product.

### INTRODUCTION

Direct capture of CO₂ from air plays an immediate and crucial role in achieving net-negative emissions and limiting ambient greenhouse gas, but high costs continue to hinder economic viability and challenges with long-term storage of gaseous CO₂ hamper sequestration capacity.¹ A major challenge of direct air capture (DAC) rises from the low feed CO₂ concentration (˜0.04% in ambient air) that requires special sorption materials designed to exhibit strong affinity to CO₂ to achieve effective capture, which are typically coupled with high energy penalty in cycling processes. As such, commercial DAC efforts are confronted with difficulty in value generation and additional cost in storage and transportation. Comparatively, a strategy where DAC and conversion of CO₂ are integrated provides a promising solution and opens up a broader scope of DAC output, increasing its value to possibly orders of magnitudes.

The first step involves the development of sorbent candidates focusing on simultaneously improving capturing capacity, selectivity, kinetics, energy consumption, and cycling stability. A series of small-molecular functionalized metal-organic frameworks (MOFs) and/or covalent organic frameworks (COFs) that exhibit CO₂ capturing capacity from humid ambient air with a moderate regeneration energy cost have been developed.²⁻⁵ The high CO₂ selectivity endows the production of high-purity CO₂ gas, which is promising for feeding into next processes without extra cost. By integration with downstream processes, the pressurization, storage, and transportation of CO₂ are circumvented, and the energy flow during the capture can be better managed and optimized.

Autotrophic microorganisms do not require a fixed source of organic carbon and predominantly obtain carbon through intracellular CO₂ fixation. A bevy of research has been undertaken to understand the cellular and enzymatic processes that govern CO₂ fixation, in particular with an eye toward its application for the capture and conversion of anthropogenic CO₂.^{6, 7} Acetogenic autotrophic bacteria have been identified as potential ‘living’ biocatalysts for their ability to convert CO₂ not only to biomass but also acetate as a byproduct of the Wood-Ljungdahl pathway.^{8, 9} For example, Sporomusa ovata (S. ovata), an acetogenic bacterial strain, can take up electrons directly from a poised cathode or oxidize H₂ as sources of reducing equivalents for the conversion of CO₂ to acetate.^{10, 11} We have demonstrated that acetate can serve as a cornerstone carbon molecule and be used as a feedstock in secondary bioprocesses in which heterotrophic microbes (e.g. Escherichia coli) generate higher-value carbon molecules such as biopolymers, biofuels and fine chemicals.^{12, 13} Inorganic electrocatalysts, especially metal nanocrystals, have emerged as an attractive system for CO₂ reduction due to their ability to attain high conversion rates.^{14, 15} However, biological catalysts remain unparalleled in terms of long-term stability, product specificity, and their ability for self-generation and repair.^{16, 17}

Various methods of electro-microbial CO₂ fixation have been proposed, where (as in the example above) autotrophic organisms use electrochemically generated mediator molecules (e.g. H₂) as an energy source to biologically fix CO₂. However, none have been coupled with DAC systems, and most have only been demonstrated at a bench scale. Therefore, engineering-level concerns and process environmental impacts had previously not been studied to a sufficient degree.

We have developed bioelectrochemical models of various electro-microbial production schemes in order to predict reasonable titers, energy efficiencies, and productivities achievable by these systems.^{18, 19} Additionally, we have extended these bioelectochemical models to process models of integrated Direct Air Capture and Electro-Microbial Production (DAC-EMP) systems and performing Life Cycle Impact Assessments (LCIA) of those processes. These models are modular, and can be adapted for various direct air capture methods, EMP schemes, and can model production of biomass or various value-added products.

Our modeling indicated that an integrated DAC-EMP process relying on an acetogenic autotrophy described above will have lower carbon footprints than traditional bioprocesses if the electricity is supplied through renewable sources (e.g. solar and wind), as well as a significantly lower land occupation footprint. Additionally, integrated DAC-EMP processes combined with hydrothermal liquefaction of the generated biomass provides long-term net-negative carbon sequestration. In addition to evaluating a DAC-EMP process in general, this model provides a useful tool in the engineering design of such a process. For example, we have found an important tradeoff between the energy efficiency of the bioelectrochemical reactor and its productivity. The process and life cycle impact model, however, provides a single metric (global warming potential) for which engineering design choices may be evaluated.

Traditional bioprocesses that produce fuels, plastics, and commodity chemicals could replace petroleum-based processes. However, most biotechnological strategies for such production rely on heterotrophic systems that require land- and resource-intensive production of feedstocks from corn and sugarcane. Hence, our strategy for coupling the direct air capture (DAC) of CO₂ with electro-microbial production has the potential to substantially alleviate the carbon footprint and land use of bioprocesses when paired with a renewable energy source. By carefully balancing the ratio of value-added product and sequestration material produced, our modeling predicted processes that achieve both economic viability and net carbon sequestration.

We have now developed, practically implemented and disclose here such systems and processes, and in particular modular direct air capture and bioelectrochemical conversion of CO₂ to chemicals and biomacromolecules like industrial protein production.

Aspects of this invention were published as Cestellos-Blanco et al. Front. Microbiol., Vol. 12, 28 Jul. 2021, Production of PHB From CO2-Derived Acetate With Minimal Processing Assessed for Space Biomanufacturing.

### SUMMARY OF THE INVENTION

We have invented and disclose a modular integrated system to directly capture and convert CO₂ from ambient air to modifiable value-added products. Our system serves as a platform of carbon removal and in a broader scheme and larger scale, aids in making supply chains carbon negative or neutral. The customizable system offers chemicals, enzymes, fuels, pharmaceuticals, or biopolymers to an array of industrial partners.

In an aspect the invention provides systems, processes, and methods for capturing CO₂ from ambient or near-ambient air, electro-biochemical conversion of CO₂ into multi-carbon products and the production of biomass.

The invention provides an integrated system which, in the first step, directly captures CO₂ from air by a sorbent. In some embodiments, the sorbents are fully or partially comprised of functionalized metal-organic framework or covalent organic framework material. The CO₂, typically enriched and purified, is converted to an upgradeable organic carbon intermediate by autotrophic microorganisms (e.g acetate by Sporomusa ovata) in an optimized bioreactor. The autotrophs derive energy from the oxidation of electrochemically generated reducing equivalents (e.g. H₂). The organic carbon intermediate is then used as a feedstock in a second bioreactor by a suitable metabolically engineered microbe (e.g. Escherichia coli) to generate (a) value-added biochemical products and/or (b) carbon-dense solid/liquid biomaterial that can be more easily stored at centennial time-scales. Our modular system, which can be tailored for a number of applications, establishes a viable approach to directly capture and convert ambient CO₂ to value-added products. Exemplary reaction products include fuels, biopolymers, pharmaceuticals, industrial enzymes, commodity chemicals and biomass. In embodiments, the integrated process comprises components which can be understood by reference to the following description taken in conjunction with the accompanying figures.

In an aspect the invention comprises components 1-3:

Component 1. Direct Air Capture of CO₂. The first component comprises a system process where a gaseous CO₂-containing stream is introduced to an adsorbent material, where CO₂ is selectively captured and released into an enriched stream. In some embodiments, the solid adsorbent is fully or partially comprised of MOF or COF materials. In some variations, the material comprises a MOF comprising periodically repeating secondary building units (SBUs) linked by organic linkers. In some other variations, the material comprises a COF comprising periodically repeating organic moieties linked by covalent bonds. In other variations, CO₂ sorbents other than MOFs or COFs are used to replace MOF/COF in the foregoing description, including but not limited to zeolites, amine-functionalized silica, functionalized cellulose, porous polymers, etc. In some variations, the active sorbents are supported by substrates that are comprised of metal, oxide, hydroxide, ceramic, salt, polymer, and/or biological or biologically derived materials. In some embodiments, the CO₂ is released and collected through the regeneration of the sorbent with variation of temperature, pressure, humidity, or other physicochemical parameters. In some embodiments, the heat is provided through direct heating (steam, electric, light), and in some other embodiments, the heat is provided through induction heating. In some aspects, the CO₂ is collected on one side of a membrane separator. In some variations, such processes are repeated in a cycling manner

Component 2. (Bio)-Electrochemical conversion of CO₂ to heterotroph feedstock. The high-purity CO₂ stream is converted to an organic carbon molecule (such as but not limited to acetate or formate) that serves as a feedstock to the downstream bioreactor, typically either by a bacterial lithoautotrophic process (e.g. to produce acetate) or an electrochemical process (e.g. to produce formate) catalyzed by a metal catalyst.

Component 3. Biochemical upgrading of organic carbon feedstock to value-added and storable products. The organic carbon molecule (e.g. acetate, formate) generated in component 2 is upgraded by a metabolically engineered heterotrophic or organoautotrophic bacteria. This microbe converts the organic carbon into a wide range of products including but not limited to hydrocarbons, small molecules, proteins and enzymes. The waste biomass produced alongside products of interest may be converted into carbon-dense solid and liquid material via hydrothermal liquefaction, pyrolysis, or a related thermochemical process which may be sequestered at centennial time scales.

In an aspect the invention provides an integrated, modular system for direct air capture (DAC) and electro-microbial production (EMP) for bioelectrochemical conversion of CO₂, the system comprising: (a) a solid absorbent configured to directly capture CO₂ from air; (b) a first bioreactor configured to receive enriched and purified CO₂ from the absorbent and convert the CO₂ to an upgradeable organic carbon intermediate by an autotrophic microorganism (e.g acetate by Sporomusa ovata), wherein the autotrophic microorganism derives energy from oxidation of electrochemically-generated reducing equivalents (e.g. H₂); and (c) a second bioreactor configured to receive the organic carbon intermediate from the first bioreactor for use as a feedstock by a heterotrophic microorganism (e.g., Escherichia coli) to generate a value added product such as a fuel, biopolymer, pharmaceutical, industrial enzyme, commodity chemical or biomass.

In embodiments:
the solid adsorbent comprises a metal-organic framework (MOF) or covalent organic framework (COF) material;
the solid adsorbent comprises a composite or mixture of adsorbents, or further comprises a binder or substrate;
the heterotrophic microorganism is metabolically engineered
the system further comprising a gas-solid contactor to host the solid adsorbent that processes air, compressed air, or similar gas mixtures with dilute CO₂ (e.g. 0 to 5%);
the first and second bioreactors are contained in a common vessel, forming a combined bioreactor;
the first and second bioreactors are contained in separate vessels;
the system is configured such that the second bioreactor is obtained by inoculating the first bioreactor with heterotrophic microorganism;
the second bioreactor is one of multiple second bioreactors, mechanically, operably and switchably interfaced with the first bioreactor, such as in a spoke-hub, manifold or carousel configuration;
the system is configured to operate in batch or continuous production mode;
the system is autonomously powered;
the autotrophic microorganism is metabolically engineered;
the autotrophic microorganism is an acetogen such as Sporomusa ovata, Clostridium ljungdahlii, Clostridium drakei, or Moorella thermoacetica;
the heterotrophic microorganism is naturally acetotrophic (e.g. by a native acetate-assimilation pathway such as the glyoxylate bypass);
the heterotrophic microorganism is a non-naturally acetotrophic microorganism engineered or adapted to assimilate acetate as a carbon and energy source either through rational methods (e.g. genetically introducing enzymes required for acetate assimilation) or adaptive/evolutionary methods (e.g. through adaptive laboratory evolution or mutant library generation and screening);
the heterotrophic microorganism is an acetotrophic baterium such as Escherichia coli, Cupriavidus necator, Cupriavidus basilensis, Psuedomonas putida, Pseudomonas aeruginosa, Psuedomonas fluorescens, Bacillus subtilis, Bacillus licheniformis, Corynebacterium glutamicum, Rhodobacter sp., Clostridium sp., Aeromonas sp.; mixotrophic algae such as Chlorella sp., Chlamydomonas sp.; or fungi/yeasts such as Yarrowia lipolytica, Aspergillus oryzae, Cryptococcus curvatus;
the heterotrophic microorganism is an acetotrophic microbe that produces a naturally occurring metabolite or biomolecule that is the desired product, or is genetically engineered to produce the desired end product;
the product is a fuel such as isobutanol, n-butanol or ethanol;
the product is a bioplastic such as polyhydroxyalkanoate and associated co-polymers; a commodity chemical/precursor such as glycerol, 3-hydroxypropionic acid, lactic acid, malonic acid, propionic acid, serine, acetoin, aspartic acid, fumaric acid, malic acid, succinic acid, threonine, arabinitol, glutamic acid, itaconic acid, proline, xylitol, xylonic acid, aconitic acid, citric acid, glucaric acid, lysine, sorbitol, glucose, fructose, sucrose; small-molecule pharmaceuticals such as artemisinin, benzylpenicillin, streptomycin, doxorubicin; proteins such as in use as industrial enzymes, therapeutic proteins, vaccines; biomass for use as a single-cell protein, fertilizer, or to be thermocatalytically upgraded to liquid bio-oil and/or solid biochar via hydrothermal liquefaction, pyrolysis, or a related process; or some combination of the aforementioned products;
the heterotrophic microorganism is either a natural formatotroph (e.g. Cupriavidus necator) or a non-naturally formatotrophic microorganism engineered or adapted to assimilate acetate as a carbon and energy source either through rational methods (e.g. genetically introducing enzymes required for formate-driven carbon fixation or formate assimilation) or adaptive/evolutionary methods (e.g. through adaptive laboratory evolution or mutant library generation and screening), and is fed formate/formic acid electrochemically generated from the captured carbon dioxide;
the heterotrophic microorganism is formatotroph that produces a naturally occurring metabolite or biomolecule that is the desired product, or is genetically engineered to produce the desired end product;
the heterotrophic microorganism is formatotroph and the product is any of the products or product categories listed above;
the heterotrophic microorganism is either a natural Knallgas (aerobic hydrogen-oxidizing) bacterium (e.g. Cupriavidus necator) or a non-naturally Knallgas microorganism engineered or adapted to fix carbon dioxide using hydrogen gas as an energy source either through rational methods (e.g. genetically introducing enzymes required for hydrogen-driven carbon fixation) or adaptive/evolutionary methods (e.g. through adaptive laboratory evolution or mutant library generation and screening) and the system contains a single hydrogen gas/carbon dioxide-fed bioreactor;
the heterotrophic microorganism is a Knallgas microbe that produces a naturally occurring metabolite or biomolecule that is the desired product, or is genetically engineered to produce the desired end product; and/or
the microorganism is a Knallgas bacterium and the product is any of the products or product categories listed above.

In an aspect the invention provides a method comprising operating a disclosed system for direct air capture (DAC) and electro-microbial production (EMP) for bioelectrochemical conversion of CO₂.

The invention encompasses all combinations of the particular embodiments recited herein, as if each combination had been laboriously recited.

### BRIEF DESCRIPTION OF THE DRAWINGS

FIG. 1. Schematic representation (top) and process flow diagram (bottom) of an embodiment of the three-stage integrated DAC-EMP system.

FIGS. 2A, 2B. Results from one full bioprocess integration. (A) Cell growth and acetate generation by S. ovata in DM9 (B) Biomass yield, acetate consumption, and poly(3-hydroxybutyrate) (PHB) accumulation by C. basilensis bioacetate rich spent DM9.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or and polypeptide sequences are understood to encompass opposite strands as well as alternative backbones described herein. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

In an aspect the invention provides integrated systems combining direct air capture-electro-microbial production, as well as their designing and implementing strategies depending on capturing scenario, energy source, desired products, and application scale. The system is generally comprised of three components: (1) direct air capture of CO₂, (2) (bio)electrochemical conversion of CO₂ to an organic carbon and (3) biochemical upgrading of the organic carbon to value-added and storable products. In some variations, the three steps are correspondingly realized in one of three mutually interfaced devices; in some variations, two or three steps are combined in one device. In some variations, peripheral devices are used for energy supply, feed supply, product storage and transfer, and/or biomass treatment.

In an aspect, the energy input of the system is provided with sustainable energy including but not limited to solar, wind, geothermal, waste-to-power sources. In some variations, the system generates such energy by itself; in some variations, the system is connected to output of such plants or a power grid. In some variations, the system utilizes input of heat instead of electricity from industrial exhaust, water cooling, HVAC waste heat, waste-to-power heat, or other similar sources.

In Component 1, provided is a system subprocess wherein a gaseous CO₂-containing stream is introduced to a solid adsorbent, to which CO₂ is selectively captured and released into an enriched stream.

In some aspects, the feed stream of the component comprises a low concentration of (0 to 5%) CO₂ and is introduced in a passive or forced manner In some variations, the stream is ambient air, fan/turbine/pump-compressed air, HVAC (heating, ventilation and air conditioning) exhaust, closed-space atmosphere, or CO₂-lean gas streams in industrial processes. In some variations, the stream contains moisture.

In some aspects, the solid adsorbent is fully or partially comprised of MOF or COF materials. In some variations, MOF or COF are physically blended with or chemically bound to substrates that are comprised of metal, oxide, hydroxide, ceramic, salt, polymer, and/or biological materials.

In some variations, the MOF comprises periodically repeating SBUs linked by organic linkers. In some variations, the SBUs comprise of one or more metals or metal-containing complexes. In some variations, the SBUs form one-dimensional extended rod-like chains or distinct multinuclear metal clusters. In certain variations, the organic linkers comprise one or more linear/V-shaped ditopic moieties, trigonal tritopic moieties, rectangular or tetrahedral tetratopic moieties, hexagonal or trigonal prismatic or trigonal antiprismatic or octahedral hexatopic moieties, or octagonal or rectangular or cubic or tetragonal antiprismatic octatopic moieties, or dodecatopic moieties. In certain variations, side chains are present that contain organic or inorganic structures, and are attached to SBUs through coordination bonds, or to linkers through covalent bonds. In some embodiments, the linkers contain at least two carboxylate, hydroxamate, pyrazolate, triazolate, tetrazolate, hydroxide, sulfide, amide, phosphonate, phosphinate, or imidazolate groups that directly coordinate with at least one metal.

In some other variations, the COF comprises periodically repeating organic moieties linked by covalent bonds in stacked two-dimensional or three-dimensional extended structures. In certain variations, the organic moieties are one or covalent-bound combination of more linear/V-shaped ditopic moieties, trigonal tritopic moieties, rectangular or tetrahedral tetratopic moieties, hexagonal or trigonal prismatic or trigonal antiprismatic or octahedral hexatopic moieties, or octagonal or rectangular or cubic or tetragonal antiprismatic octatopic moieties, or dodecatopic moieties. These COFs are interconnected with covalently bound multitopic atom group termed linkages. In certain variations, side chains are present that contain organic or inorganic structures, and are attached to organic moieties or the linkages through covalent bonds. In some embodiments, the linkages are one or combination of boronic anhydride, boronate ester, borosilicate, imine, enamine, hydrazone, pyrazine, olefin, acrylonitrile, ester, amide, amine, triazine, squaric acid, tetrahydroquinoline, dihydroquinoline, or pyridine.

In some variations, the invention provides multivariate MOFs or COFs (MTV-MOFs or MTV-COFs) of two or more structurally or compositionally different SBUs, organic linkers, or organic moieties are present in symmetrically equivalent sites while the overall framework connectivity remains unchanged.

In some embodiments, the framework comprises alkylamine functionalized and configured for capturing CO₂ through chemisorption, specifically, where primary or secondary alkyl amines are present in the SBUs, organic linkers, or side chains in MOFs or in the organic moieties or linkages in COFs. In some variations of such MOFs or COFs, the alkylamine species capture CO₂ from the contacting gas stream through reaction to form carbamic acid, carbamate, or bicarbonate intermediates, which can be released by applying moderate heating, vacuum, or a combination of both. The capacity of this process is not impaired by the presence of moisture, but in some variations increased instead. In variations, the active site is selected from alkyl alcohols, vicinal diols, thiolates, amides, hydroxides, and their derivatives. In some other variations, physisorptive materials instead of chemisorptive materials are used. In a particular embodiment, a MOF possessing fluorine atoms in a narrow, polar channel exhibiting physisorptive properties to CO₂ is used. The MOFs and COFs are configured to be particularly suitable for capturing CO₂ in practical DAC-relevant conditions, utilizing but not limited to this capture-and-release mechanism.

In other variations, CO₂ sorbents other than MOFs or COFs are used to replace MOF/COF in the foregoing description, including but not limited to zeolites, molecular cages, amine-impregnated silica, amine-functionalized silica, functionalized cellulose, porous or non-porous polymers, porous coordination polymers, functionalized carbon, functionalized oxides or hydroxides, layered or porous metal/semimetal carbides, nitrides, oxides, silicides, phosphides, and chalcogenides, layered and/or porous carbon nitrides, ionic liquids, liquid amine or amine-containing small molecules or polymers, biomolecules, or their physical or chemical composites.

In some aspects, the disclosed adsorbent is applied into a sorption device such as a packed bed, fluidized bed, surface contactor, or combination or variations of such forms. In some variations, the disclosed adsorbent is deployed in a membrane separator.

In some aspects, the CO₂ is released and collected through the regeneration of the sorbent with variation of temperature, pressure, humidity, or other physicochemical parameters. In some aspects, the CO₂ is collected on a side of a membrane separator. In some variations, such processes are repeated in a cycling manner In some variations, the process is executed continuously without an apparent cycling pattern.

In certain variations, wherein the process captures water from the gas source when moisture is present. In some embodiments, the water is collected through condensation, membrane separation, or other methods. In some embodiments, the water is fed into the component 2 or 3, or used for parts of supplying components. In some embodiments, the water is directly taken as output as a product.

Components 2 and 3 center around the bioelectrochemical conversion of carbon dioxide directly captured from the atmosphere into value-added products and/or a dense and stable form of carbon that can be sequestered at centennial timescales under ambient temperature and pressure. There are therefore many combinations with which one may carry out this process.

There are multiple organisms and reactor setups that may be used for the bioelectrochemical conversion of the CO₂ to the end product. In each iteration, CO₂, electricity, and water are the primary feedstocks for the system. In a preferred design, electrolysis of water is used to produce H₂. This H₂, along with the captured CO₂, is fed into an anaerobic bioreactor containing an acetogenic bacteria such as Sporomusa ovata, Clostridium ljungdahlii, Clostridium drakei, etc. which convert a large fraction of the CO₂ to acetate (a relatively small portion of the carbon dioxide is fixed by the acetogen to produce biomass and other metabolites). The acetate produced in the first bioreactor is recovered and fed into a second aerobic bioreactor containing a heterotrophic aerobe, such as Escherichia coli, which consumes the acetate to produce biomass and/or a value-added product.

In an alternative variation, H₂ is produced by electrolysis of water as before. The H₂, the captured CO₂, and oxygen are fed into an aerobic bioreactor containing an aerobic hydrogen-oxidizing bacteria (also known as knallgas bacteria) such as Cupriavidus necator or Rhodopseudomonas palustris.²⁰ In this system, the captured carbon dioxide, using the electrolytically-derived hydrogen gas as an energy source, can be directly converted to the desired end product. In this variation, components 2 and 3 are integrated into a single process.

In an alternative variation for the bioelectrochemical conversion, captured CO₂ is fed to an electrolyzer which converts water and CO₂ to formic acid and oxygen gas. The formic acid is then fed into an aerobic bioreactor containing either an organism naturally capable of formatotrophic growth (such as Cupriavidus necator) or an organism which may be genetically engineered to be able to grow on formate (such as Escherichia coli).^{21,22} This microbe then biochemically converts the formic acid into the desired end product.

Each embodiment of the bioelectrochemical conversion can be adapted for various products of interest. In one variation, the system produces biomass without any additional value-added product. The biomass produced in the system is then converted to some combination of bio-oil (a crude-like liquid oil) and bio-char (a solid charcoal). These products sequester the carbon that is fixed by the microbes in the system, and are stable at ambient conditions for centennial timescales. This conversion can be achieved by hydrothermal liquefaction, pyrolysis, or a related thermochemical process. Hydrothermal liquefaction is better suited for wet biomass, which will be produced in this process, and is therefore the better candidate for long-term carbon sequestration.²³ Alternatively, the system can be designed primarily to produce a value-added product. The range of products possible in this system range from industrial enzymes, biofuels, bioplastics, commodity chemicals, and pharmaceuticals.²⁴ In these processes, the system can be optimized to divert as much carbon as possible from the captured carbon dioxide to the desired end product. The system will produce waste biomass as a byproduct, and in these systems, the waste biomass generated will undergo conversion to bio-oil and bio-char as described previously.

In a preferred design, we use the first method of bioelectrochemical conversion listed, in which captured carbon dioxide and electrolytically-derived hydrogen gas are first converted to acetate by an acetogenic microbe which is then fed into a downstream bioreactor containing a heterotrophic microbe. This method has a few key advantages over the alternatives. First, this system enables the greatest modularity. The acetogen would require little to no engineering as it is the same regardless of the desired product. The second reactor can contain E. coli, which has already been engineered to produce a myriad of potential products, including enzymes, pharmaceuticals, fuels, plastics, and various other chemical products, allowing minimal changes to the overall system. The other systems, which require microbes with a fairly unique metabolism, typically require further engineering in order to produce multiple products. Cupriavidus necator for example has already been engineered to produce a few value-added products. However, these are fewer in number than have been produced in E. coli. Secondly, hydrogen electrolysis is much more energy efficient than formate electrolysis, providing an advantage over the formate-based system. Thirdly, the Wood-Ljungdahl present in acetogens is a more energy efficient carbon fixation pathway than the Calvin Cycle seen in many Knallgas and formatotrophic organisms. Lastly, as acetogens are obligate anaerobes, the hydrogen feed stream can be fed without oxygen, and therefore cannot combust, which is intrinsically safer than the other hydrogen-based system.

As stated before, various products may be produced in this system. Sequestered bio-oil and bio-char are advantageously carbon negative products. Producing a fuel such as isobutanol provides a larger market potential, with have a higher net carbon footprint. Industrial enzymes have a lower annual demand than fuels, however still have a substantial global demand Additionally, producing industrial enzymes through this process is more likely to become carbon negative over its life cycle than producing biofuels due to a larger portion of the fixed carbon being diverted towards biomass.

Example: Production of PHB from CO₂-Derived Bioacetate with Minimal Processing for Space Biomanufacturing

Here, we present an integrated two-module process for the production of PHB from CO₂. An autotrophic S. ovata process converts CO₂ to acetate which is then directly used as the primary carbon source for PHB production by C. basilensis. We have designed and optimized our process to require no purification or filtering of the cell culture media between microbial production steps. 10.4 mmol acetate L⁻¹ day⁻¹ were generated from CO₂ by S. ovata in the optimized media. Subsequently, 12.54 mg PHB L⁻¹ hour⁻¹ were produced by C. basilensis in the unprocessed media with an overall carbon yield of 11.06% from acetate.

Optimization of Base Medium for Bioprocess

We firstly cultured S. ovata autotrophically in balch-type 25 mL culture tubes with an 80/20% H₂/CO₂ headspace. In these conditions acetate generation rate amounts to 10.4 mmol L⁻¹ day⁻¹. We selected 25 mM acetate as the feedstock concentration for C. basilensis. This titer of bioacetate was achieved within the first 48-72 hours of the autotrophic S. ovata culture. However, the S. ovata culture could reach a concentration of 50-60 mM bioacetate in 5-7 days without adjusting for pH which decreases due to acetate accumulation. The S. ovata cultures were stopped at 25 mM bioacetate and aerated which rendered the S. ovata inactive. C. basilensis was then inoculated in the spent S. ovata cultures. The increase in biomass as detected by OD₆₀₀ was used to monitor the ability of C. basilensis to grow and use the CO₂-derived bioacetate. The baseline OD resulting from the presence of S. ovata biomass in the spent medium was deducted. As compared to C. basilensis grown in fresh DM9 (per L: 1 g NH₄Cl, 7 g Na₂HPO₄, 3.9 g NaH₂PO₄-2H₂O, 1.47 ml of 10 g/LCaCl₂-2H₂O, 10 ml of 0.1M MgSO₄-7H₂O) which is the recommended C. basilensis culture medium, with synthetic acetate the biomass yield in the spent S. ovata medium was only 50% within the timeframe of the experiment. We employed a second control with cultured C. basilensis in fresh S. ovata medium (no prior S. ovata) with synthetic acetate. This culture achieved the same biomass yield as the one in the spent S. ovata medium indicating that bioacetate is not incompatible with C. basilensis but rather that a component in the S. ovata medium inhibits C. basilensis growth. In a second medium optimization experiment, we again cultured S. ovata autotrophically until the bioacetate concentration reached 25 mM. The spent S. ovata medium was diluted twofold with fresh DM9 containing synthetic acetate at 35 mM, 25 mM and 0 mM. Thus the mixed media which were inoculated with C. basilensis contained total acetate concentrations of 30 mM, 25 mM and 12.5 mM respectively. The biomass yield in the 25 mM culture equaled that of C. basilensis in fresh DM9 in the prior experiments. The biomass yields in the 30 mM and 12.5 mM were congruent with the available acetate. While adding fresh DM9 clearly enhanced C. basilensis growth, the cultures containing spent S. ovata medium had a 20 hour longer lag phase. The peak optical densities were only reached in twice the length of time. Furthermore, it is burdensome to formulate two sets of media. Therefore, we attempted to grow S. ovata directly in deoxygenated DM9. We found that a 10% (v/v) inoculum is necessary when culturing S. ovata in DM9 whereas normally only a 5% (v/v) would be required. Next, we employed culturing controls in which we added the recommended S. ovata medium components to a DM9 base including reducing reagent, vitamins and carbonate. Favorably S. ovata not only grows well in DM9 but also generates a similar amount of acetate as in the recommended S. ovata medium. Significant decreased growth and acetate generation is only detected in the carbonate containing DM9 which could be a result of the increased osmotic pressure of the saline medium.

Process Integration for PHB Production

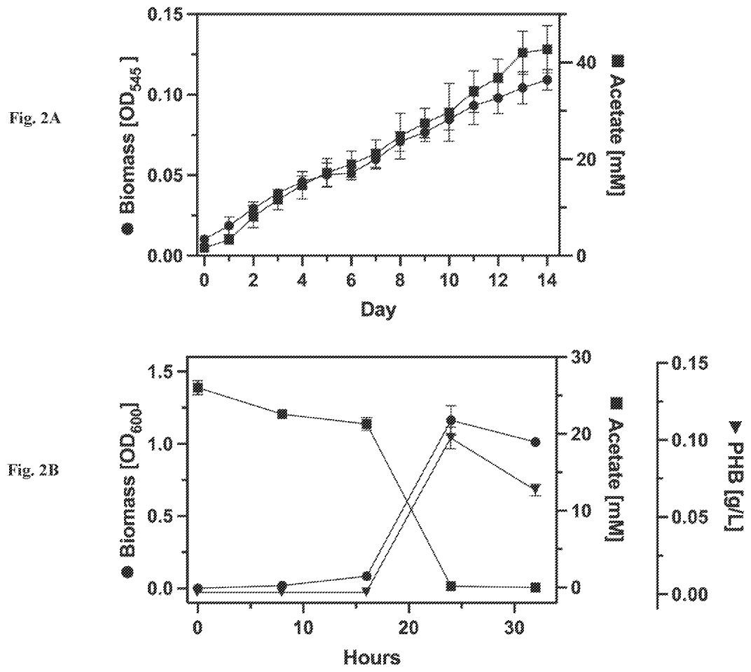

Before inoculating with S. ovata, we deoxygenated the DM9 medium. In order to gauge PHB productivity, we scaled up our process from 25 mL tubes to 1L balch-type bottles each with 270 mL of culture medium (FIG. 2a). There was a significant scale-up associated loss in acetate productivity with the cultures reaching 25 mM only after eight days. The S. ovata cultures were grown for 14 days and the acetate concentration reached 42 mM. We proceeded to culture C. basilensis in the aerated spent DM9 which was diluted to 25 mM (FIG. 2b). As expected, the acetate concentration decreases as biomass accumulates. It is worth noting that the biomass yield achieved in this experiment was decidedly greater than that obtained in fresh DM9. Additionally, there is no protracted lag phase with the use of 100% DM9 as the base medium. Next, we determined that PHB correlates well with biomass production and we calculated a PHB generation rate of 12.6 mg L⁻¹ hour⁻¹ with an overall 11.06% carbon yield from acetate. After 24 hours there is a decrease in the PHB concentration which correlates with the complete consumption of acetate.

Qualitative Cell Population Analysis

We fixed culture samples at the 0 hour time point just after inoculating the spent DM9 with C. basilensis. As S. ovata had achieved approximately 0.12 OD₅₄₅, we expected to mostly detect S. ovata. On the SEM micrographs rod-shaped S. ovata is abundantly visible with very few C. basilensis cells. Additionally, we fixed culture samples at the 24 hour time point after the exponential growth of C. basilensis. At this stage, C. basilensis cells clearly dominate the landscape. C. basilensis closely co-exist with inactive S. ovata cells, indicating that S. ovata cells are at least innocuous to C. basilensis and underscoring the ability to use spent S. ovata medium directly with minimal processing.

### REFERENCES

1. Negative Emissions Technologies and Reliable Sequestration. Negative Emissions Technologies and Reliable Sequestration (National Academies Press, 2019). doi:10.17226/25259
2. Trickett, C. A. et al. The chemistry of metal-organic frameworks for CO2 capture, regeneration and conversion. Nat. Rev. Mater. 2, 1-16 (2017).
3. Ding, M., Flaig, R. W., Jiang, H. L. & Yaghi, O. M. Carbon capture and conversion using metal-organic frameworks and MOF-based materials. Chemical Society Reviews 48, 2783-2828 (2019).
4. Flaig, R. W. et al. The Chemistry of CO2 Capture in an Amine-Functionalized Metal-Organic Framework under Dry and Humid Conditions. J. Am. Chem. Soc. 139, 12125-12128 (2017).
5. Diercks, C. S. & Yaghi, O. M. The atom, the molecule, and the covalent organic framework. Science 355, (2017).
6. Claassens, N. J., Sousa, D. Z., Dos Santos, V. A. P. M., De Vos, W. M. & Van Der Oost, J. Harnessing the power of microbial autotrophy. Nature Reviews Microbiology 14, 692-706 (2016).
7. Lovley, D. R. Bug juice: Harvesting electricity with microorganisms. Nature Reviews Microbiology 4, 497-508 (2006).
8. Drake, H. L. & Daniel, S. L. Physiology of the thermophilic acetogen Moorella thermoacetica. Res. Microbiol. 155, 869-883 (2004).
9. Fast, A. G. & Papoutsakis, E. T. Stoichiometric and energetic analyses of non-photosynthetic CO 2-fixation pathways to support synthetic biology strategies for production of fuels and chemicals. Curr. Opin. Chem. Eng. 1, 380-395 (2012).
10. Moller, B., Obmer, R., Howard, B. H., Gottsehalk, G. & Hippe, H. Sporomusa, a new genus of gram-negative anaerobic bacteria including Sporomusa sphaeroides spec. nov. and Sporomusa ovata spec. nov. *. Arch Mierobiol 139, (1984).
11. Nevin, K. P., Woodard, T. L., Franks, A. E., Summers, Z. M. & Lovley, D. R. Microbial Electrosynthesis: Feeding Microbes Electricity To Convert Carbon Dioxide and Water to Multicarbon Extracellular Organic Compounds. MBio 1, (2010).
12. Liu, C. et al. Nanowire-bacteria hybrids for unassisted solar carbon dioxide fixation to value-added chemicals. Nano Lett. 15, 3634-3639 (2015).
13. Su, Y. et al. Close-Packed Nanowire-Bacteria Hybrids for Efficient Solar-Driven CO2 Fixation. Joule 4, 800-811 (2020).
14. Ross, M. B. et al. Designing materials for electrochemical carbon dioxide recycling. Nat. Catal. 2, 648-658 (2019).
15. Lu, Q. & Jiao, F. Electrochemical CO2 reduction: Electrocatalyst, reaction mechanism, and process engineering. Nano Energy 29, 439-456 (2016).
16. Cestellos-Blanco, S., Zhang, H., Kim, J. M., Shen, Y. & Yang, P. Photosynthetic semiconductor biohybrids for solar-driven biocatalysis. Nat. Catal. 3, 245-255 (2020).
17. Kornienko, N., Zhang, J. Z., Sakimoto, K. K., Yang, P. & Reisner, E. Interfacing nature's catalytic machinery with synthetic materials for semi-artificial photosynthesis. Nat. Nanotechnol. 13, 890-899 (2018).
18. Abel, A. J., Hilzinger, J. M., Arkin, A. P. & Clark, D. S. Systems-informed genome mining for electroautotrophic microbial production. bioRxiv 2020.12.07.414987 (2020). doi:10.1101/2020.12.07.414987
19. Abel, A. J. & Clark, D. S. A Comprehensive Modeling Analysis of Formate-Mediated Microbial Electrosynthesis**. ChemSusChem 14, 344-355 (2021).
20. Yu, J. & Munasinghe, P. Gas Fermentation Enhancement for Chemolithotrophic Growth of Cupriavidus necator on Carbon Dioxide. Fermentation 4, 63 (2018).
21. Grunwald, S. et al. Kinetic and stoichiometric characterization of organoautotrophic growth of Ralstonia eutropha on formic acid in fed-batch and continuous cultures. Microb. Biotechnol. 8, 155-163 (2015).
22. Kim, S. J., Yoon, J., Im, D. K., Kim, Y. H. & Oh, M. K. Adaptively evolved Escherichia coli for improved ability of formate utilization as a carbon source in sugar-free conditions. Biotechnol. Biofuels 12, 207 (2019).
23. Process Design and Economics for the Conversion of Algal Biomass to Hydrocarbons: Whole Algae Hydrothermal Liquefaction and Upgrading|PNNL. Available at: https://www.pnnl.gov/publications/process-design-and-economics-conversion-algal-biomass-hydrocarbons-whole-algae. (Accessed: 15 Mar. 2021)
24. Chen, X. et al. Metabolic engineering of Escherichia coli: A sustainable industrial platform for bio-based chemical production. Biotechnology Advances 31, 1200-1223 (2013).

1. An integrated, modular system for direct air capture (DAC) and electro-microbial production (EMP) for bioelectrochemical conversion of CO₂, the system comprising:
(a) a solid absorbent configured to directly capture CO₂ from air;
(b) a first bioreactor configured to receive enriched and purified CO₂ from the absorbent and convert the CO₂ to an upgradeable organic carbon intermediate by an autotrophic microorganism (e.g acetate by Sporomusa ovata), wherein the autotrophic microorganism derives energy from oxidation of electrochemically-generated reducing equivalents (e.g. H₂); and
(c) a second bioreactor configured to receive the organic carbon intermediate from the first bioreactor for use as a feedstock by a heterotrophic microorganism (e.g., Escherichia coli) to generate a value added product such as a fuel, biopolymer, pharmaceutical, industrial enzyme, commodity chemical or biomass.

2. The system of claim 1, wherein the solid adsorbent comprises a metal-organic framework (MOF) or covalent organic framework (COF) material.

3. The system of claim 1, wherein the solid adsorbent comprises a composite or mixture of adsorbents, or further comprises a binder or substrate.

4. The system of claim 1, wherein the heterotrophic microorganism is metabolically engineered.

5. The system of claim 1, further comprising a gas-solid contactor to host the solid adsorbent that processes air, compressed air, or gas mixtures, with dilute CO₂ (0 to 5%).

6. The system of claim 1, wherein the first and second bioreactors are contained in a common vessel, forming a combined bioreactor.

7. The system of claim 1, wherein the first and second bioreactors are contained in separate vessels.

8. The system of claim 1, wherein the system is configured such that the second bioreactor is obtained by inoculating the first bioreactor with the heterotrophic microorganism.

9. The system of claim 1, wherein the second bioreactor is one of multiple second bioreactors, mechanically, operably and switchably interfaced with the first bioreactor, such as in a spoke-hub, manifold or carousel configuration.

10. The system of claim 1, wherein the system is configured to operate in batch or continuous production mode.

11. The system of claim 1, wherein the system is autonomously powered.

12. The system of claim 1, wherein the autotrophic microorganism is metabolically engineered.

13. The system of claim 1, wherein the autotrophic microorganism is an acetogen such as Sporomusa ovata, Clostridium ljungdahlii, Clostridium drakei, or Moorella thermoacetica.

14. The system of claim 1, wherein the heterotrophic microorganism is naturally acetotrophic by a native acetate-assimilation pathway such as the glyoxylate bypass.

15. The system of claim 1, wherein the heterotrophic microorganism is a non-naturally acetotrophic microorganism engineered or adapted to assimilate acetate as a carbon and energy source either through rational methods, particularly genetically introducing enzymes required for acetate assimilation, or adaptive/evolutionary methods, particularly through adaptive laboratory evolution or mutant library generation and screening.

16. The system of claim 1, wherein the heterotrophic microorganism is an acetotrophic baterium such as Escherichia coli, Cupriavidus necator, Cupriavidus basilensis, Psuedomonas putida, Pseudomonas aeruginosa, Psuedomonas fluorescens, Bacillus subtilis, Bacillus licheniformis, Corynebacterium glutamicum, Rhodobacter sp., Clostridium sp., Aeromonas sp.; mixotrophic algae such as Chlorella sp., Chlamydomonas sp.; or fungi/yeasts such as Yarrowia lipolytica, Aspergillus oryzae, Cryptococcus curvatus.

17. The system of claim 1, wherein the heterotrophic microorganism is an acetotrophic microbe that produces a naturally occurring metabolite or biomolecule that is the desired product, or is genetically engineered to produce the desired end product.

18. The system of claim 1, wherein the product is a fuel such as isobutanol, n-butanol or ethanol.

19. The system of claim 1, wherein the product is a bioplastic such as polyhydroxyalkanoate, such as PHB, and associated co-polymers.

20. The system of claim 1, wherein the product is a commodity chemical/precursor such as glycerol, 3-hydroxypropionic acid, lactic acid, malonic acid, propionic acid, serine, acetoin, aspartic acid, fumaric acid, malic acid, succinic acid, threonine, arabinitol, glutamic acid, itaconic acid, proline, xylitol, xylonic acid, aconitic acid, citric acid, glucaric acid, lysine, sorbitol, glucose, fructose, sucrose.

21. The system of claim 1, wherein the product is a small-molecule pharmaceuticals such as artemisinin, benzylpenicillin, streptomycin, doxorubicin.

22. The system of claim 1, wherein the product is a protein such as in use as industrial enzymes, therapeutic proteins, vaccines.

23. The system of claim 1, wherein the product is a biomass for use as a single-cell protein, fertilizer, or to be thermocatalytically upgraded to liquid bio-oil and/or solid biochar via hydrothermal liquefaction, pyrolysis, or a related process.

24. The system of claim 1, wherein the heterotrophic microorganism is either a natural formatotroph (e.g. Cupriavidus necator) or a non-naturally formatotrophic microorganism engineered or adapted to assimilate acetate as a carbon and energy source either through rational methods, particularly genetically introducing enzymes required for formate-driven carbon fixation or formate assimilation, or adaptive/evolutionary methods, particularly through adaptive laboratory evolution or mutant library generation and screening, and is fed formate/formic acid electrochemically generated from the captured carbon dioxide.

15. The system of claim 1, wherein the heterotrophic microorganism is a non-naturally acetotrophic microorganism engineered or adapted to assimilate acetate as a carbon and energy source either through rational methods, particularly genetically introducing enzymes required for acetate assimilation, or adaptive/evolutionary methods, particularly through adaptive laboratory evolution or mutant library generation and screening.

25. The system of claim 1, wherein the heterotrophic microorganism is formatotroph that produces a naturally occurring metabolite or biomolecule that is the desired product, or is genetically engineered to produce the desired end product.

26. The system of claim 1, wherein the heterotrophic microorganism is formatotroph and the product is any of the products or product categories listed above.

27. The system of claim 1, where the heterotrophic microorganism is either a natural Knallgas (aerobic hydrogen-oxidizing) bacterium (e.g. Cupriavidus necator) or a non-naturally Knallgas microorganism engineered or adapted to fix carbon dioxide using hydrogen gas as an energy source either through rational methods, particularly genetically introducing enzymes required for hydrogen-driven carbon fixation, or adaptive/evolutionary methods, particularly through adaptive laboratory evolution or mutant library generation and screening, and the system comprises a single hydrogen gas/carbon dioxide-fed bioreactor.

28. The system of claim 1, wherein the heterotrophic microorganism is a Knallgas microbe that produces a naturally occurring metabolite or biomolecule that is the desired product, or is genetically engineered to produce the desired end product.

29. The system of claim 1, wherein the microorganism is a Knallgas bacterium and the product is any of the products or product categories listed above.

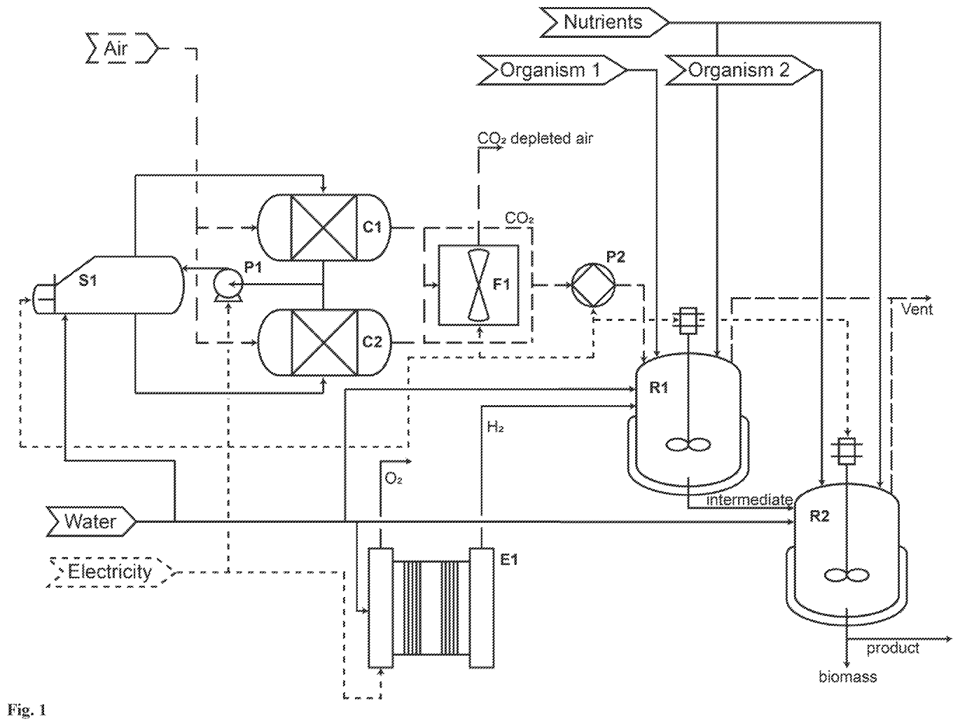

30. The system of claim 1, configured as shown in FIG. 1.

31. A method comprising operating a system of claim 1 providing direct air capture (DAC) and electro-microbial production (EMP) for bioelectrochemical conversion of CO₂.
